(19) United States
(12) Patent Application Publication
(10) Pub. No.: US 2026/0131069 A1
(43) Pub. Date: May 14, 2026

(54) RECIPROCATING PUMP SEGMENT

(71) Applicant: CareFusion 303, Inc., San Diego, CA (US)

(72) Inventor: Richard O’CONNOR, Limerick (IE)

(21) Appl. No.: 18/946,819

(22) Filed: Nov. 13, 2024

(51) Int. Cl. A61M 5/142

(52) U.S. Cl. CPC A61M 5/14228

(57) ABSTRACT

An apparatus for preserving an elasticity of a pumping segment of an infusion set into an infusion pump. The apparatus includes a rigid tubing support surface and one or more rotating members each comprising a keyway configured to receive one or more respective support keys fixedly attached to the pumping segment such that a rotation of a compressible tubing of the pumping segment is prevented from rotating with respect to the respective support keys. The rotating members at least partially rotate the pumping segment about a central axis of the pumping segment by causing the one or more respective support keys to at least partially rotate, while constraining the pumping segment in a predetermined linear position between the rigid tubing support surface and a pumping mechanism of the infusion pump and while the pumping mechanism applies a periodic compression force to the pumping segment.

### TECHNICAL FIELD

The present disclosure relates generally to intravenous infusion pump mechanisms. In particular, the present disclosure relates to operation of an infusion pump with regard to an infusion set pumping segment.

### BACKGROUND

In the medical field, infusion pumps are used often to administer accurate amounts of infusates from an external bag to a patient via an intravenous (IV) administration set. The IV administration set is used to administer fluids, medications, or blood products directly into the patient's bloodstream. It typically consists of a plastic or flexible tubing with connectors on either end—one end attaches to a fluid source such as a saline bag or medication vial, while the other end connects to a needle or catheter inserted into a vein. The IV administration set includes a pumping segment—a section of tubing in the IV administration set—that loads into the infusion pump and passes through the pump's rollers or squeeze mechanism. As the pump compresses and releases the tubing of the pumping segment in a rhythmic motion, it generates a pulsatile flow, effectively propelling fluid through the tubing of the IV administration set and into the patient's bloodstream.

### SUMMARY

As existing volumetric infusion pumps operate by compressing an elastic segment of the IV administration set against a platen to deliver fluid, the repeated mechanical compression causes the elastic segment to undergo localized plastic deformation. Initially, the elastic segment exhibits elastic behavior, allowing it to return to its original shape after each compression cycle. However, over time, the continuous stress and strain cause the material in the compressed regions to exceed its elastic limit, transitioning into plastic behavior. This plastic deformation results in permanent changes to the shape and thickness of the elastic segment, which affects its ability to maintain consistent compression force against the platen. Consequently, this transition from elastic to plastic behavior leads to a change in flow rate accuracy, causing the infusion pump to deliver fluid at a rate that may deviate from the intended or calibrated flow rate, thereby impacting the reliability and precision of fluid delivery over time.

Typically, the IV administration set is replaced approximately every 72 hours to maintain accurate fluid delivery and to minimize the risk of complications such as infection or mechanical failure due to material fatigue. However, if the elasticity of the infusion set can be preserved throughout its use, this replacement interval can be safely extended to 96 hours or more. Enhancing the material properties to resist plastic deformation and maintain consistent elasticity would not only improve flow rate accuracy over an extended period but also reduce the frequency of set changes, resulting in cost savings, reduced labor for healthcare providers, and less disruption for patients.

The present disclosure addresses the aforenoted problems by providing an apparatus for preserving an elasticity of a pumping segment of an infusion set into an infusion pump. According to various implementations, the apparatus comprises a rigid tubing support surface; and one or more rotating members each comprising a keyway configured to receive one or more respective support keys fixedly attached to the pumping segment such that a rotation of a compressible tubing of the pumping segment is prevented from rotating with respect to the one or more respective support keys, the one or more rotating members being configured to, when each support key of the pumping segment is received into the corresponding keyway, at least partially rotate the pumping segment about a central axis of the pumping segment by causing the one or more respective support keys to at least partially rotate, while constraining the pumping segment in a fixed linear position between the rigid tubing support surface and a pumping mechanism of the infusion pump and while the pumping mechanism applies a periodic compression force to the pumping segment. Other aspects include corresponding systems and methods for implementation of the corresponding apparatus and its features.

It is understood that other configurations of the subject technology will become readily apparent to those skilled in the art from the following detailed description, wherein various configurations of the subject technology are shown and described by way of illustration. As will be realized, the subject technology is capable of other and different configurations and its several details are capable of modification in various other respects, all without departing from the scope of the subject technology. Accordingly, the drawings and detailed description are to be regarded as illustrative in nature and not as restrictive.

### BRIEF DESCRIPTION OF THE DRAWINGS

For a better understanding of the various described implementations, reference should be made to the Description of Implementations below, in conjunction with the following drawings. Like reference numerals refer to corresponding parts throughout the figures and description.

FIG. 1A depicts a perspective view of an example infusion pump showing an infusion set in place within the infusion pump, according to various aspects of the subject technology.

FIG. 1B depicts an example patient care unit shown, according to various aspects of the subject technology.

FIG. 2 depicts an example pumping mechanism of an infusion pump, according to various aspects of the subject technology.

FIG. 3 depicts an example cam phase diagram corresponding to a fluid delivery cycle of an infusion pump, according to various aspects of the subject technology.

FIG. 4A depicts a cutaway view of an apparatus for preserving an elasticity of a pumping segment of an infusion set into an infusion pump, according to aspects of the subject technology. FIGS. 4B and 4C depict a cutaway view of alternative configurations of the apparatus for preserving an elasticity of a pumping segment of an infusion set into an infusion pump, according to aspects of the subject technology.

FIG. 5 depicts an example process for operating a pumping segment of an infusion set in an infusion pump to preserve an elasticity of the pumping segment, according to aspects of the subject technology.

FIG. 6 is a conceptual diagram illustrating an example electronic system for operating a pumping segment of an infusion set in an infusion pump to preserve an elasticity of the pumping segment, according to aspects of the subject technology.

### DESCRIPTION

Reference will now be made to implementations, examples of which are illustrated in the accompanying drawings. In the following description, numerous specific details are set forth, in order to provide an understanding of the various described implementations. However, it will be apparent to one of ordinary skill in the art that the various described implementations may be practiced without these specific details. In other instances, well-known methods, procedures, components, circuits, and networks have not been described in detail so as not to unnecessarily obscure aspects of the implementations.

As will be described further, the subject technology includes a reciprocal mechanism which rotates an IV administration set loaded into an infusion pump with respect to the pump fingers and platen around its central axis, effectively changing the location of the points of maximum stress across a section or the entirety of the set circumference.

In some implementations, two mechanical keys are rigidly fixed to the extremes of the pump segment, one upstream and one downstream. The mechanical keys interface into dedicated keyways, adjacent to ends of the pump's pumping mechanism, to fix the position of the segment with respect to the pumping mechanism, preventing rotation about the segment's axis. The linear distance of the set between the keyways may also be fixed. As will be described further, the keyways may be of one piece or of multiple pieces, but are connected in such a way that they can rotate in unison about the axis of the segment, while being constrained by the pump body. The keyways are connected to a mechanism which incrementally rotates the segment about its axis throughout the infusion in a reciprocating manner. While a complete rotation may occur in some implementations, according to various implementations described herein, the rotation is a partial rotation that rocks and/or oscillates back and forth. The actuation of the mechanism may borrow from existing linear or rotational movements of the pumping mechanism, or may be drive by an additional actuator.

Some of the advantages of embodiments consistent with the present disclosure include the compression of the set being no longer localized on two points (or lines) on one side of the segment. Accordingly, more of the segment's elastic material is being utilized, thereby reducing the degradation of the material, and thus reducing the corresponding degradation in the rate accuracy over time, in addition to a reduction in spallation. The typical administration set change interval may thus be increased (e.g., >72 hours).

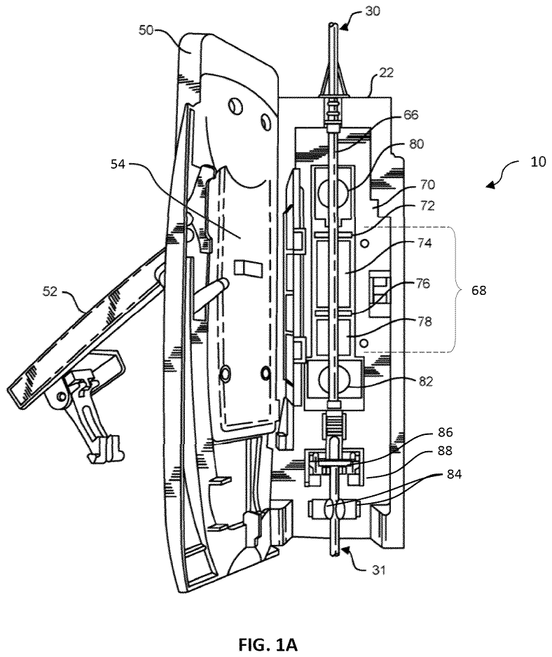

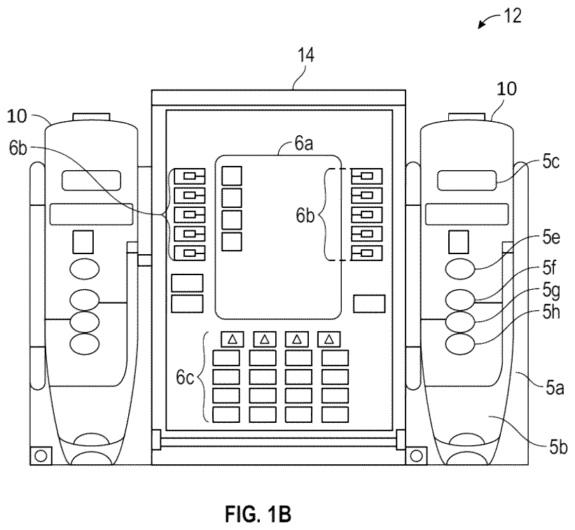

FIG. 1A depicts a perspective view of an example infusion pump showing an infusion set in place within the infusion pump, according to various aspects of the subject technology. An infusion system for parenteral infusion of a medical fluid to a patient comprises a pump unit 10, a major part of which comprises a housing or casing 22 which accommodates, in manner known per se, a cam system (not shown) controlling a plurality of fingers of a pumping mechanism, an electric motor and associated gearing, driving said cam mechanism, and further accommodates electronic control and processing circuitry for controlling such motor and processing signals from pressure sensors etc. provided on the unit. The pump unit, as shown, may also comprise an electronically operated display, an alarm light, an input keyboard or other manually operated controls, all in manner known per se.

As shown in FIG. 1A, the infusion pump 10 (aka an “infusion device”) is shown in perspective view with the front door 50 open, showing the upstream fluid line 30 (e.g., portion between fluid container and the infusion pump 30) and downstream fluid line 31 (e.g., portion between the infusion pump 31 and a patient) in operative engagement with the infusion pump 10. An infusion set tubing may include a continuous fluid conduit formed from the upstream fluid line 30 to the downstream fluid line 31, extending from a respective fluid supply to a patient, through which fluid is acted upon by the pump to move fluid downstream to the patient. The infusion pump 10 directly acts on a pumping segment 66 of the infusion set that passes through the pumping mechanism 70 of the pump. Specifically, the pumping mechanism 70 acts as the flow control device of the pump to move fluid though the conduit. The depicted references 30, 31, 66 may be used to describe herein portions of one continuous fluid line or, in some implementations, may individually describe portions that are fluidly connected together to form a continuous fluid line. The upstream and downstream fluid lines and/or tube 30, 31, 66 may also be coupled to a pump cassette or cartridge that is configured to be coupled to the pump 10.

As shown in FIG. 1A, the face plate 50 which may be opened to reveal the internal loading mechanism for an infusion set. Within the housing of the infusion pump (e.g., behind the door or face place), the infusion pump includes a pumping mechanism 70 including a group of serially-aligned pumping elements 72, 74, 76, 78 configured to compress an elongated compressible channel of the pumping segment 66, when loaded within the pumping mechanism of the pump. The pumping mechanism includes a group of serially-aligned pumping elements (e.g., occluders and/or pumping finger(s)) configured to compress the elongated compressible channel (e.g., an IV tubing segment) against a rigid tubing support surface 54 when the pumping segment 66 is loaded within the pumping mechanism.

The infusion set includes an intermediate section of the resiliently compressible tubing 66, for example of silicone rubber and, in some implementations, upper and/or lower fittings which each tubing section may be connected respectively with a respective upper line 30 and with the lower line 31. In use, each upper line 30 extends upwardly to a source of the medical fluid to be administered whilst the lower line 31 extends from the infusion pump to an infusion needle or the like inserted into the patient. In use, the infusion set 66 is extended across the face or deck of the pump unit so that its fittings (not labeled) are received in respective brackets respectively and so that the tubing segment extends over a pumping mechanism. In the depicted example, the pumping mechanism 70 includes a four finger pump assembly 72, 74, 76, 78. In the depicted example, the infusion set is fitted in place in this fashion whilst the door 50 is in the open position. After the infusion line has been so fitted, the door 50 may be moved to the closed position and is secured by a catch 52 which may include a lever mounted on the outer edge of the door. When the door is closed, the rigid support surface 54 is locked below the pump assembly so that the pumping segment 66 may be compressed against the surface 54 by elements 72-78.

The four finger pump assembly 72, 74, 76, 78 includes respective fingers (and/or occluders) that are moveable by a cam system inwards and outwards from the face or deck of the pump to compress a respective tubing segment against a counter surface or anvil to propel fluid within the infusion line. In order to make it easier to maintain sterile conditions, these fingers may be covered by a thin flexible membrane, (not shown), sealed at its edges with respect to the deck. The fingers of the pump assembly periodically press the flexible resilient tubing against the counter surface 54 which, as described previously, may be configured on an opposite side, for example, on an inner portion of the door 50.

The type of pumping mechanism may vary, including the number of fingers in the pumping mechanism. In the depicted example, the pumping mechanism 70 includes an upstream occluding element or finger 72, a primary pumping element or finger 74, a downstream occluding element or finger 76, and a secondary pumping element or finger 78. The pumping mechanism (and mechanisms used in other linear peristaltic pumps) operate by sequentially pressing on a segment of the fluid conduit by means of the cam-following pumping elements (e.g., pumping fingers and valve fingers) 72, 74, 76, and 78, which in the depicted example make a four finger pump assembly. Each element may be sequentially activated by a respective cam lobe on a camshaft to apply a downward compression against tubing 66, to move the fluid in the tubing 66 downstream. Intermediate pumping mechanism 74 may include multiple intermediate elements or fingers (not shown) that sequentially activate according to positioning of the cam lobes. In some implementations, the pressure is applied in sequential locations of the conduit, beginning at the upstream end of the pumping mechanism, and working toward the downstream end. At least one finger is always pressing hard enough to occlude the conduit. As a practical matter, one finger does not retract from occluding the tubing until the next one in sequence has already occluded the tubing; thus, at no time is there a direct fluid path from the fluid supply to the patient. The operation of peristaltic pumps including four finger pumps is well known to those skilled in the art and no further operational details are provided here.

An upstream pressure sensor 80 may also be included in the pump 10. The upstream pressure sensor may be mounted to pumping mechanism 70 or located adjacent and upstream in relation to the pumping mechanism 70 between a fluid supply and the pumping mechanism 70, so that the connection of the correct fluid supply with the correct pump may be verified before any fluid is pumped to the patient. A downstream pressure sensor 82 is also included in the example infusion pump 10 at a downstream location with respect to the pumping mechanism, that is, at a location between the patient and the flow control device, so that the connection of the correct fluid supply with the correct pump may be verified before any fluid is pumped to the patient. Downstream pressure sensor 82 may be used to detect a pressure change adjacent to occluder 76 and/or downstream finger 78, to determine whether these elements are functioning properly.

The pump 10 also includes an air-in-line (AIL) sensor 84. In some implementations, air-in-line sensor 84 may be mounted at a bottom end of chassis 22. air-in-line sensor 84 may include two sensing elements disposed on opposite sides of a slot through which the infusion tubing 66 passes. The air-in-line sensor 84 includes a signal (e.g., an ultrasound signal) traveling across the tubing that includes the infusion fluid). Accordingly, infusion tubing 66 should be seated appropriately so that the ultrasound signal in air-in-line sensor 84 traverses the fluid.

In some implementations, the infusion set 66 may include a cartridge 86 that clips into a compartment 88 of the pump for proper positioning. In the depicted example, the cartridge 86 includes a flow stop that is received into a corresponding compartment 88 in the casing 22 above the air-in-line sensor 84. In this regard, the flow stop cartridge plugs into the compartment and facilitates alignment of the fluid line 66 with the pumping components, and further facilitates alignment of the downstream line 31 with the air-in-line sensor 84.

FIG. 1B depicts an example patient care unit 12 shown, according to various aspects of the subject technology. FIG. 1B shows two functional infusion pumps 10 (e.g., “infusion pump modules”) mounted at either side of a main frame infusion controller 14, and the displays and control keys of each, with the main frame infusion controller 14 being capable of programming both infusion pumps. The infusion pump includes a door 5a and a handle 5b that operates to lock the door in a closed position for operation and to unlock and open the door for access to the internal pumping and sensing mechanisms and to load administration sets for the pump. When the door 5a is open, the tube can be connected with the pump 10. When the door 5a is closed, the tube is brought into operating engagement with the pumping mechanism, the upstream and downstream pressure sensors, and the other equipment of the pump. A display 5c, such as an LED display, is located in plain view on the door in this embodiment and may be used to visually communicate various information relevant to the pump 10, such as alert indications (e.g., alarm messages). Control keys 5e-h may exist for programming and controlling operations of the infusion pump as desired. In some implementations, the control keys may be presented as interactive elements on the display 5c (e.g., touchscreen display). The main frame and/or functional module may also include audio alert equipment in the form of a speaker (not shown).

The main frame infusion controller 14 of the patient care unit 12 includes a display 6a for visually communicating various information, such as the operating parameters of a connected pump and alert indications and alert messages, and control keys 6b and 6c for selecting and/or setting control parameters and/or options for controlling the patient care unit 12 and connected modules. The main frame infusion controller 14 may also include a speaker to provide audible alerts. In some implementations, the display 6a may be implemented as a touchscreen display. In such implementations, the control keys 6b may be omitted or reduced in number by providing corresponding interactive elements via a graphical user interface presented via the display 6a. In some implementations, each control key 6b (or 6c) may select a corresponding option displayed in display 6b.

The main frame infusion controller 14 may include a communications system (not shown) with which the main frame infusion controller 14 may communicate with external equipment such as a medical facility server or other computer and with a portable processor, such as a handheld communication device or a laptop-type of computer, or other information device that a clinician may have to transfer information as well as to download drug libraries to a functional module 10. The communication module may be used to transfer access and interaction information for clinicians encountering the main frame infusion controller or device coupled therewith (e.g., pump 10 or bar code scanner). The communications system may include one or more of a radio frequency (RF) system, an optical system such as infrared, a BLUETOOTH™ system, or other wired or wireless system. The bar code scanner and communications system may alternatively be included integrally with the infusion pump 10, such as in cases where a main frame infusion controller is not used, or in addition to one with the main frame infusion controller 14. Further, information input devices need not be hard-wired to medical instruments, information may be transferred through a wireless connection as well. Additionally, other types of modules may be connected to the pump modules or to the main frame infusion controller such as a syringe pump module, patient controlled analgesic module, end tidal CO₂ monitoring module, oximeter monitoring module, or the like.

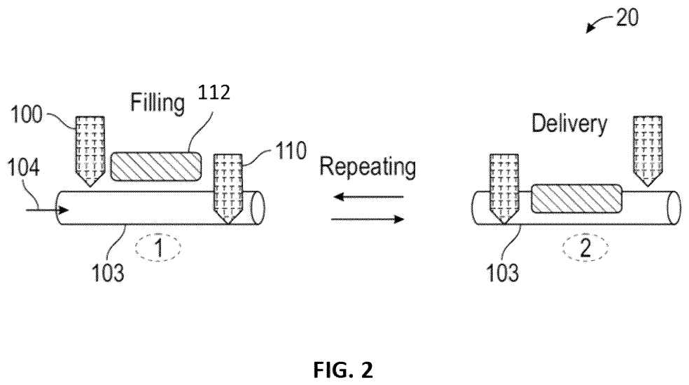

FIG. 2 depicts an example pumping mechanism 70 of an infusion pump 10, according to various aspects of the subject technology. A typical medical pump for IV infusion delivery has two occluders, a first occluder 100 located upstream and a second occluder 110 located downstream, with a plunger 112 (e.g., pumping element 74) in between. The occluders and plunger coordinate with each other in programmable, sequential steps, controlled by a cam shaft to have two phases: 1) a filling phase, and 2) a delivery phase. The occluders move fluid in a tubing 103 of pumping segment 66 by sequentially compressing the tubing, thereby causing a flow in a direction 104 according to the particular compression sequence of the occluders.

During the medication infusion process, in the filling phase, the upstream occluder 100 lifts to suck the medication into the tubing segment, which creates a pause, followed by the delivery phase to push the fluid out. These sequences can repeat through multiple cycles. To specify, when the plunger of a single plunger/tubing design is lifted from the tubing segment during the filling phase, there will be a disruption in the continuous infusion process.

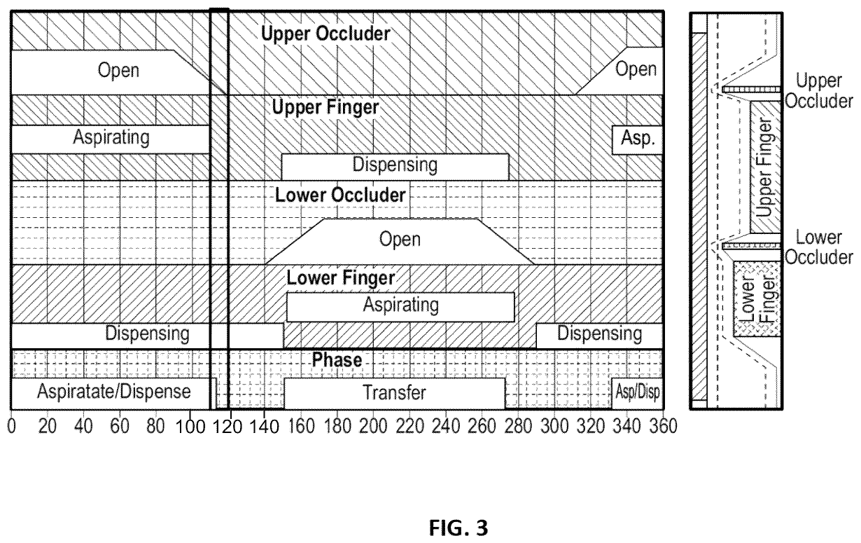

FIG. 3 depicts an example cam phase diagram corresponding to a fluid delivery cycle of an infusion pump, according to various aspects of the subject technology. Each row depicted in FIG. 3 corresponds to a respective pumping element 72, 74, 76, 78 of the pump's pumping mechanism, and illustrates an example pumping function of the element according to a complete cam rotation (360°). That is which pumping elements are closing (compressing the tube) and which elements are opening, thereby creating an aspiration phase and a dispensing phase.

In the depicted example, initially, from 0°to 90°, the upper element, e.g., upper occluder 72, is open, while the lower occluder remains closed. The upper element 72 completes closing at about 120°; however, the fluid tubing may be sufficiently compressed to stop aspiration of the fluid at about 110°. The lower element, e.g., lower occluder 78 remains closed until the cam reaches 140°. While the upper occluder 72 is open—until it begins to close at about 90°—the upper finger 74 is aspirating. The upper element closes between 90-120°, and the lower element begins to open at 140°. At this point, the lower finger 76 will begin to deliver the fluid. In the depicted example, delivery begins at about 145°, with about 5°rotation accounting for the time to decompress the tubing.

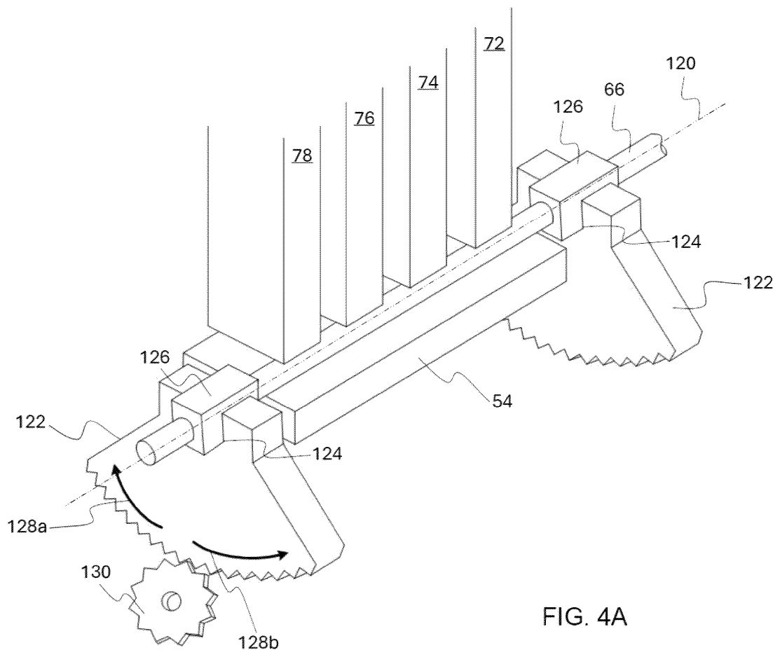

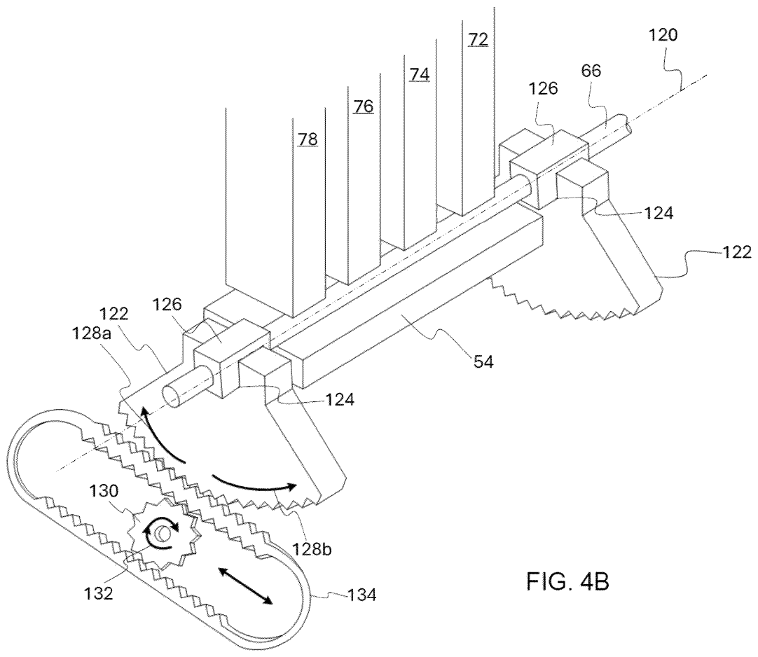

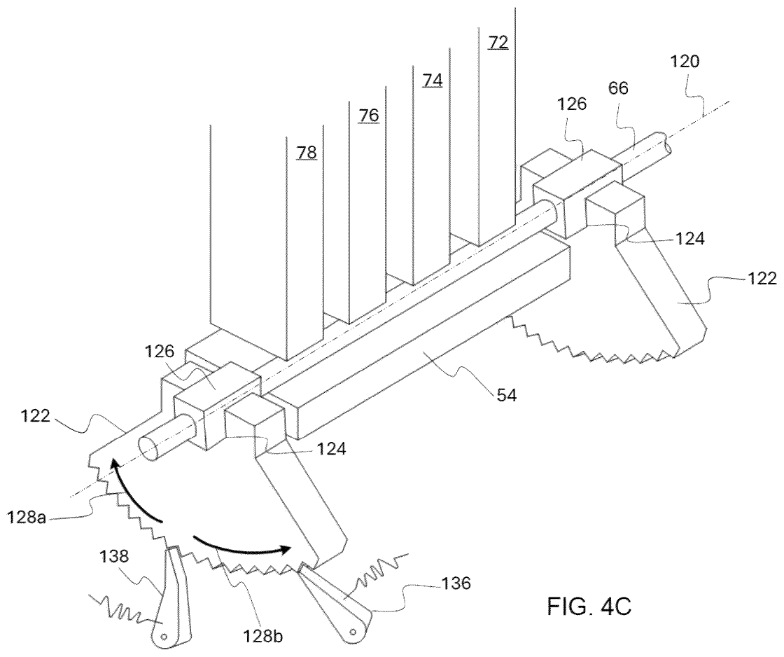

FIG. 4A depicts a cutaway view of an apparatus for preserving an elasticity of a pumping segment of an infusion set into an infusion pump, according to aspects of the subject technology. The depicted example is illustrative of the internal components of a pumping mechanism 70 within a pump being observable along the infusion tubing path. With reference to the example of FIG. 1, the location of the support surface 54 and fingers 72-28 may be interchanged such that the support surface 54 of the apparatus may be implemented within the door 50 of the pump 10.

According to various implementations, the apparatus includes a rigid tubing support surface 54, against which the pumping elements 72-78 may compress the pumping segment 66 loaded within the pump 10. As described previously the support surface 54 may be integrated within the door 50 of the pump such that, when the door is closed, the surface 54 is positioned to support the tubing segment 66 while the pumping mechanism 70 and its elements 72-28 are acting upon the segment during an infusion.

The apparatus includes one or more rotating members 122 each comprising a keyway 124 configured to receive one or more respective support keys 126 fixedly attached to the pumping segment 66 such that a rotation of the compressible tubing of the pumping segment 66 is prevented from rotating with respect to the one or more respective support keys 126 but rotation of the one or more rotating members 122 will cause rotation of the pumping segment 66 by virtue of rotating the one or more support keys 126. In some implementations, the pumping segment includes two support keys 126, one at each end of the pumping mechanism. In this regard, the support keys 126 may be integrated with the pumping segment 66 such that the IV administration set is provided with the support keys at both ends of the pumping segment 66. The keys may be mechanically fixed with couplings to the tubing segment 66 or, in some implementations, may be chemically or temperature bonded. Accordingly, in some implementations, two rotating members 122 are spaced apart such that each keyway 126 of each rotating member 122 receives a respective one of the two support keys fixedly attached to the pumping segment 66 when the pumping segment 66 is loaded into the infusion pump 10, as depicted in FIG. 4A.

The one or more rotating members 122 are configured to, when each support key 126 of the pumping segment 66 is received into the corresponding keyway 124, at least partially rotate the pumping segment 66 about a central axis 120 of the pumping segment by causing the one or more respective support keys 126 to at least partially rotate, while constraining the pumping segment 66 in a fixed (e.g., predetermined) linear position (along axis 120) between the rigid tubing support surface 54 and the pumping mechanism 70 of the infusion pump 10 and while the pumping mechanism applies periodic compression force to the pumping segment 66.

The linear distance between the keyways may be fixed. In some implementations, the distance between the keyways may change over time, or may be adjusted, according to certain parameters. For example, as the pumping segment 66 of the infusion set relaxes over time the distance between the keys 126 could increase so that a constant tension is kept in the pumping segment. In some implementations, the keys 126, and the keyways 124 may be adjusted by the user. In such implementations, the adjustment may be manual or automatically performed by a drive mechanism responsive to parameters input at control keys 6 or via a touch screen associated with the infusion device.

In some implementations, the one or more rotating members are operatively coupled to a cam associated with the pumping mechanism (e.g., via one or more gears) such that the one or more rotating members rotate in coordination with a periodic compression action of the pumping mechanism 70 upon the pumping segment. That is, the rotating members may rotate according to a predetermined rotation scheme that corresponds to a range of phase angles shown in FIG. 3. In some implementations, the rotating member(s) are configured to rock back and forth to cause the pumping segment to partially rotate back and forth (128a, 128b) about the central axis 120 of the pumping segment 66.

In some implementations, the rate of the rotation of the rotating members may be fixed at a predetermined rate of rotation. In some implementations, the rate can be varied based on one or more factors, including flow rate, type of infusate, line pressure (e.g., as measured by a pressure sensor 80, 82), etc. The processor of the pump may index a lookup table by one or more of these factors to identify a predetermined rate of rotation and then control and/or adjust the rotation based on the identified rate. According to various implementations, the rotating members rotate in unison. In some implementations, the rotating members may rotate independently, for example, in a predetermined pattern. For example, during a first infusion cycle, a first keyway 124 (e.g., the downstream keyway) is rotated X-degrees clockwise, while the upstream is static. Then during the next cycle the other keyway (e.g., the upstream keyway) is rotated X-degrees clockwise, while the downstream is static.

As depicted in FIG. 4A, a gear 130 may be operatively coupled to at least one of the one or more rotating members 122 to cause the one or more rotating members to rotate in a first direction 128 a until a first threshold rotation is reached and then to rotate in an opposite direction 128 b until a second threshold rotation is reached in the opposite direction, and then to continually repeat the back and forth rotation until compression actions performed by the pumping mechanism upon the pumping segment are terminated. In some implementations, gear(s) 130 is driven by the cam associated with the pumping mechanism. In some implementations, the gear(s) 130 is driven by an electronic motor that is controlled by a processor associated with the pump's electronic subsystems (e.g., within the pump 10 or controller 14). Accordingly, the processor may or may not synchronize the rotation of the rotating members 122 with the pumping mechanism. For example, the rotating members may be driven independently of the pumping mechanism.

In some implementations, the gear(s) 130 (or other mechanism described herein) is configured to incrementally rotate the rotating member(s) in the first direction by a predetermined amount (e.g., a fixed number of degrees) commensurate with a predetermined number of compression actions performed by the pumping mechanism 70 until the first threshold rotation is reached and then to incrementally rotate the one or more rotating members in an opposite direction by the predetermined amount commensurate with another predetermined number of compression actions until the second threshold rotation is reached in the opposite direction. The system may then continually repeat the back and forth rotation until the compression actions are terminated. In some implementations, the rotating member(s) are rotated a step of X degrees (e.g., 1 degrees) after a set period of time (e.g., each minute, hour, ect.). In some implementations, the rotating members are continuously rotating at a fixed rate of rotation over the set period of time.

While two rotating members 122 are shown in FIGS. 4A-4C, in some implementations, the system may include only one. In some implementations, two rotating members 122 are connected together such that a rotation of a first rotating member of the two rotating members causes the two rotating members to rotate in unison. In such implementation, the two members 122 may be connected by a bar (not shown) underneath the static platen 54. In this regard, the members 122 and bar may rock or rotate in unison under and/or partially around the platen 54. In some implementations, two rotating members (and bar) are injection molded such as to form a single part. In implementations, wherein only one gear 130 drives the members 122, only the rotating member 122 that interfaces with the gear may include teeth, whereas the other member 122 merely follows the motion of the driving member it is coupled to.

In some implementations, the pumping segment 66 is configured to rotate independently of and within the IV administration set. In this regard, each of the two support keys 126 comprises a swivel connector (e.g., a universal 360°connector) configured to allow rotation of the two support keys and the compressible tubing 130 between the two support keys 126 with respect to an upper tubing segment 30 and lower tubing segment 31 of the infusion set.

In some implementations, one or more of the support key(s) 126 is shaped to center a portion of the pumping segment 66 constrained by the support key(s) with respect to a sensor of the infusion pump. For example, the keyway corresponding to the support key may include a matching shape to secure the support key, align the portion of the pumping segment with the sensor, and align pumping segment with the pumping mechanism. For example, the sensor may be a pressure sensor 80 or pressure sensor 82, and the key may conform to the shape of the sensor. In some implementations, the keyway may include a recess in a topmost surface of the rigid surface or platen 54, which is also configured to accommodate a pressure sensor 80, 82 or a receiver or transmitter of the pressure sensor when both are present. For example, the recess may be inset within the door 50, opposite a sensor 80, 82. The keyway conforming to the sensor may include a mechanism for causing rotation of the corresponding support key while keeping it aligned. Alternatively, the keyway may remain fixed while letting the portion of the pumping segment 66 rotate freely while being constrained, with the other keyway being fixed to the pumping segment such that when the other keyway is rotated, the entire pumping segment rotates while being aligned with the sensor.

FIGS. 4B and 4C depict a cutaway view of alternative configurations of the apparatus for preserving an elasticity of a pumping segment of an infusion set into an infusion pump, according to aspects of the subject technology. In some implementations, such that depicted in FIG. 4B, the apparatus includes a rack and pinion system for rotating the one or more rotating members 122. In this configuration, a linear actuator includes a circular gear 132 that engages a linear gear (the rack) 134, and the rotational motion of the gear(s) 132 is converted to linear motion (in a line) to drive the rotating member(s) 122 (via the gear teeth of the rack interfacing with teeth of the member 122) to rotate the keyway(s) and thus rotate the pumping segment 66. Accordingly, the rack and pinion system replaces the system described with respect to FIG. 4A, having the same or similar functions as previously described with regard to FIG. 4A.

In some implementations, such as depicted by FIG. 4C, the apparatus includes a bi-directional rachet and pawl operatively coupled to at least one of the one or more rotating members 122. Accordingly, the disclosed apparatus may include a driving pawl 136 and a locking pawl 138. The driving pawl 136 may be operably connected by an arm (not shown) to the cam (not shown) associated with the pumping mechanism 70 or the previously described motor, which may cause the arm (not shown), for example, to pivot about the center of the rotating member 122, which functions as a toothed ratchet wheel. The rachet and pawl system causes the rotating member(s) 122 to incrementally rotate in a first direction by a predetermined amount commensurate with a predetermined number of compression actions performed by the pumping mechanism until a first threshold rotation is reached and then to incrementally rotate the one or more rotating members in an opposite direction by the predetermined amount commensurate with another predetermined number of compression actions until a second threshold rotation is reached in the opposite direction. For example, each compression may operate the arm to drive the driving pawl 136 one increment. In some implementations, the teeth on half of the lower edge (e.g., corresponding to direction 128 a) of the rotating member(s) may be in one direction and the other half (on the opposing side corresponding to direction 128 b) in another direction. In some implementations, there may be a driving pawl 136 and a locking pawl 138 on each side, and the system configured to switch between the two directions at a predetermined point in the rotation. In this regard, the rotating member(s) may be driven to continually rotate the back and forth rotation until the compression actions are terminated.

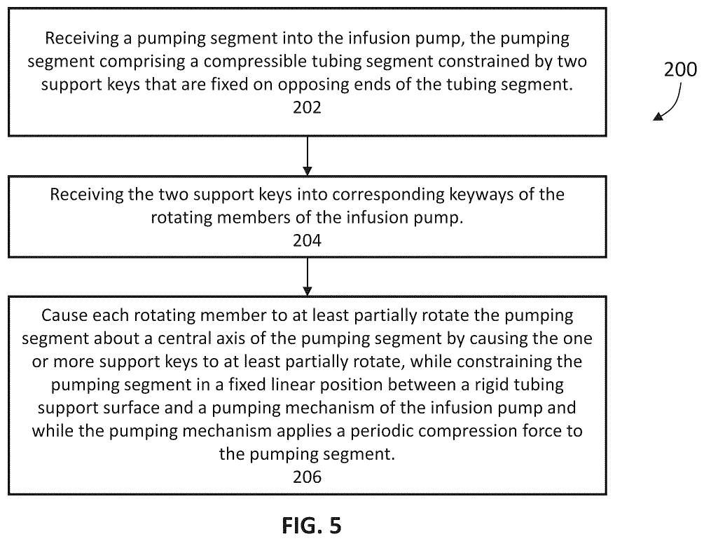

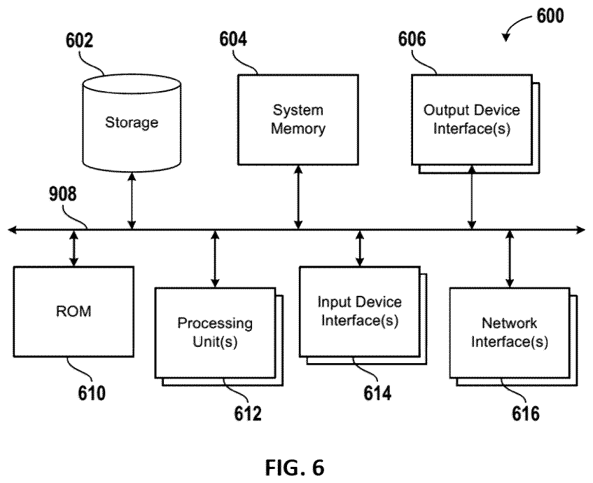

FIG. 5 depicts an example process 200 for operating a pumping segment of an infusion set in an infusion pump to preserve an elasticity of the pumping segment, according to aspects of the subject technology. For explanatory purposes, the various blocks of example process 200 are described herein with reference to FIGS. 1 through 4, and the components and/or processes described herein. The one or more of the blocks of process 200 may be implemented, for example, by the infusion pump 10 described herein. For example, one or more of the blocks may be implemented based on electromechanical and/or computer control by the pump's electronic subsystems (see, e.g., FIG. 6). For explanatory purposes, the blocks of example process 200 are described as occurring in serial, or linearly. However, the blocks of example process 200 may occur in parallel. In addition, the blocks of example process 200 need not be performed in the order shown and/or one or more of the blocks of example process 200 need not be performed.

In the depicted example, a pumping segment into the infusion pump, as shown in FIG. 1 and/or FIGS. 4A-C (202). The pumping segment includes a compressible tubing segment 66 which is constrained by two support keys, fixed on opposing ends of the compressible tubing segment. The two support keys are received into corresponding keyways of two rotating members of the infusion pump (204). Each rotating member is then caused to at least partially rotate the pumping segment 66 about a central axis 120 of the pumping segment 66 by causing the one or more respective support keys 126 to at least partially rotate, while constraining the pumping segment 66 in a fixed linear position between the previously described rigid tubing support surface 54 and pumping mechanism 70 of the infusion pump 10 and while the pumping mechanism applies a periodic compression force to the pumping segment 66. As described previously, according to various implementations, the pumping segment 66 is caused to partially rotate back and forth, periodically, in a reciprocal manner while an infusion is performed by the infusion pump 10.

Many of the above-described devices, systems and methods, may also be controlled by software processes that are specified as a set of instructions recorded on a computer readable storage medium (also referred to as computer readable medium), and may be executed automatically (e.g., without user intervention). When these instructions are executed by one or more processing unit(s) (e.g., one or more processors, cores of processors, or other processing units), they cause the processing unit(s) to perform the actions indicated in the instructions. Examples of computer readable media include, but are not limited to, CD-ROMs, flash drives, RAM chips, hard drives, EPROMs, etc. The computer readable media does not include carrier waves and electronic signals passing wirelessly or over wired connections.

The term “software” is meant to include, where appropriate, firmware residing in read-only memory or applications stored in magnetic storage, which can be read into memory for processing by a processor. Also, in some implementations, multiple software aspects of the subject disclosure can be implemented as sub-parts of a larger program while remaining distinct software aspects of the subject disclosure. In some implementations, multiple software aspects can also be implemented as separate programs. Finally, any combination of separate programs that together implement a software aspect described here is within the scope of the subject disclosure. In some implementations, the software programs, when installed to operate on one or more electronic systems, define one or more specific machine implementations that execute and perform the operations of the software programs.

A computer program (also known as a program, software, software application, script, or code) can be written in any form of programming language, including compiled or interpreted languages, declarative or procedural languages, and it can be deployed in any form, including as a stand-alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

FIG. 6 is a conceptual diagram illustrating an example electronic system 600 for operating a pumping segment of an infusion set in an infusion pump to preserve an elasticity of the pumping segment, according to aspects of the subject technology. Electronic system 600 may be representative of a control unit and/or computing device for execution of software associated with one or more components and processes provided by FIGS. 1 through 8, including but not limited to infusion pump 10 (e.g., a processing system of controller 14 or within infusion pump 10) or a electromechanical system for opening and closing the disclosed loading tray drawer 200. Electronic system 600 may be representative of a device used in connection or combination with the disclosure regarding FIGS. 1 through 5. In this regard, electronic system 600 may be a device connected to the infusion pump 10, for example, to activate the occluders and/or pumping fingers, the cam of the pumping mechanism, or to monitor or control same. For example, system 600 may be representative of a personal computer or a mobile device such as a smartphone, tablet computer, laptop, personal digital assistant (PDA), an augmented reality device, a wearable such as a watch or band or glasses, or combination thereof, or other touch screen or television with one or more processors embedded therein or coupled thereto, or any other sort of computer-related electronic device having network connectivity specifically configured to implement one or more of the features described.

Electronic system 600 may include various types of computer readable media and interfaces for various other types of computer readable media. In the depicted example, electronic system 600 includes a bus 608, processing unit(s) 612, a system memory 604, a read-only memory (ROM) 610, a permanent storage device 602, an input device interface 614, an output device interface 606, and one or more network interfaces 616. In some implementations, electronic system 600 may include or be integrated with other computing devices or circuitry for operation of the various components and processes previously described.

Bus 608 collectively represents all system, peripheral, and chipset buses that communicatively connect the numerous internal devices of electronic system 600. For instance, bus 608 communicatively connects processing unit(s) 612 with ROM 610, system memory 604, and permanent storage device 602.

From these various memory units, processing unit(s) 612 retrieves specific instructions to execute and data to process, in order to execute the processes of the subject disclosure. The processing unit(s) can be a single processor or a multi-core processor in different implementations.

ROM 610 stores static data and instructions that are needed by processing unit(s) 612 and other modules of the electronic system. Permanent storage device 602, on the other hand, is a read-and-write memory device. This device is a non-volatile memory unit that stores instructions and data even when electronic system 600 is off. Some implementations of the subject disclosure use a mass-storage device (such as a magnetic or optical disk and its corresponding disk drive) as permanent storage device 602.

Other implementations use a removable storage device (such as a floppy disk, flash drive, and its corresponding disk drive) as permanent storage device 602. Like permanent storage device 602, system memory 604 is a read-and-write memory device. However, unlike storage device 602, system memory 604 is a volatile read-and-write memory, such as random-access memory. System memory 604 stores some of the instructions and data that the processor needs at runtime. In some implementations, the processes of the subject disclosure are stored in system memory 604, permanent storage device 602, and/or ROM 610. From these various memory units, processing unit(s) 612 retrieves instructions to execute and data to process, in order to execute the processes of some implementations.

Bus 608 also connects to input and output device interfaces 614 and 606. Input device interface 614 enables the user to communicate information and select commands to the electronic system. Input devices used with input device interface 614 include, e.g., alphanumeric keyboards and pointing devices (also called “cursor control devices”). Output device interfaces 606 enables, e.g., the display of images generated by the electronic system 600. Output devices used with output device interface 606 include, e.g., printers and display devices, such as cathode ray tubes (CRT) or liquid crystal displays (LCD). Some implementations include devices such as a touchscreen that functions as both input and output devices.

Also, as shown in FIG. 6, bus 608 also couples electronic system 600 to a network (not shown) through network interfaces 616. Network interfaces 616 may include, e.g., a wireless access point (e.g., Bluetooth or WiFi) or radio circuitry for connecting to a wireless access point. Network interfaces 616 may also include hardware (e.g., Ethernet hardware) for connecting the computer to a part of a network of computers such as a local area network (“LAN”), a wide area network (“WAN”), wireless LAN, a personal area network (“PAN”), or an Intranet, or a network of networks, such as the Internet. Any or all components of electronic system 600 can be used in conjunction with the subject disclosure.

The functions described above can be implemented in computer software, firmware, or hardware. The techniques can be implemented using one or more computer program products. Programmable processors and computers can be included in or packaged as mobile devices. The processes and logic flows can be performed by one or more programmable processors and by one or more programmable logic circuitry. General and special purpose computing devices and storage devices can be interconnected through communication networks.

Some implementations include electronic components, such as microprocessors, storage and memory that store computer program instructions in a machine-readable or computer-readable medium (also referred to as computer-readable storage media, machine-readable media, or machine-readable storage media). Some examples of such computer-readable media include RAM, ROM, read-only compact discs (CD-ROM), recordable compact discs (CD-R), rewritable compact discs (CD-RW), read-only digital versatile discs (e.g., DVD-ROM, dual-layer DVD-ROM), a variety of recordable/rewritable DVDs (e.g., DVD-RAM, DVD-RW, DVD+RW, etc.), flash memory (e.g., SD cards, mini-SD cards, micro-SD cards, etc.), magnetic and/or solid state hard drives, read-only and recordable Blu-Ray® discs, ultra density optical discs, any other optical or magnetic media, and floppy disks. The computer-readable media can store a computer program that is executable by at least one processing unit and includes sets of specific instructions for performing various operations described herein. Examples of computer programs or computer code include machine code, such as is produced by a compiler, and files including higher-level code that are executed by a computer, an electronic component, or a microprocessor using an interpreter.

While the above discussion primarily refers to microprocessor or multi-core processors that execute software, some implementations are performed by one or more integrated circuits, such as application specific integrated circuits (ASICs) or field programmable gate arrays (FPGAs) specifically configured with one or more of the features described. In some implementations, such integrated circuits execute instructions that are stored on the circuit itself.

As used in this specification and any claims of this application, the terms “computer”, “server”, “processor”, and “memory” all refer to electronic or other technological devices. These terms exclude people or groups of people. For the purposes of the specification, the terms display or displaying means displaying on an electronic device. As used in this specification and any claims of this application, the terms “computer readable medium” and “computer readable media” are entirely restricted to tangible, physical objects that store information in a form that is readable by a computer. These terms exclude any wireless signals, wired download signals, and any other ephemeral signals.

To provide for interaction with a user, implementations of the subject matter described in this specification can be implemented on a specifically configured computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of specifically configured devices can be used to provide for interaction with a user as well; e.g., feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; e.g., by sending web pages to a web browser on a user's client device in response to requests received from the web browser.

Implementations of the subject matter described in this specification can be implemented in a specifically configured computing system that includes a back end component, e.g., as a data server, or that includes a middleware component, e.g., an application server, or that includes a front end component, e.g., a client computer having a graphical user interface or a Web browser through which a user can interact with an implementation of the subject matter described in this specification, or any combination of one or more such back end, middleware, or front end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer-to-peer networks).

The computing system can include clients and servers. A client and server are generally remote from each other (e.g., physically separated) and may interact through a communication network. The relationship of client and server arises by virtue of computer programs running on the respective computers and having a client-server relationship to each other. In some implementations, a server transmits data (e.g., an HTML page) to a client device (e.g., for purposes of displaying data to and receiving user input from a user interacting with the client device). Data generated at the client device (e.g., a result of the user interaction) can be received from the client device at the server.

Those of skill in the art would appreciate that the various illustrative blocks, modules, elements, components, methods, and algorithms described herein may be implemented as electronic hardware, computer software, or combinations of both. To illustrate this interchangeability of hardware and software, various illustrative blocks, modules, elements, components, methods, and algorithms have been described above generally in terms of their functionality. Whether such functionality is implemented as hardware or software depends upon the particular application and design constraints imposed on the overall system. The described functionality may be implemented in varying ways for each particular application. Various components and blocks may be arranged differently (e.g., arranged in a different order, or partitioned in a different way) all without departing from the scope of the subject technology.

It is understood that the specific order or hierarchy of steps in the processes disclosed is an illustration of example approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

Illustration of subject technology as clauses:

Various examples of aspects of the disclosure are described as numbered clauses (1, 2, 3, etc.) for convenience. These are provided as examples, and do not limit the subject technology. Identifications of the figures and reference numbers are provided below merely as examples and for illustrative purposes, and the clauses are not limited by those identifications.
Clause 1. An apparatus for preserving an elasticity of a pumping segment of an infusion set into an infusion pump, comprising: a rigid tubing support surface; and one or more rotating members each comprising a keyway configured to receive one or more respective support keys fixedly attached to the pumping segment such that a rotation of a compressible tubing of the pumping segment is prevented from rotating with respect to the one or more respective support keys, the one or more rotating members being configured to, when each support key of the pumping segment is received into the corresponding keyway, at least partially rotate the pumping segment about a central axis of the pumping segment by causing the one or more respective support keys to at least partially rotate, while constraining the pumping segment in a predetermined linear position between the rigid tubing support surface and a pumping mechanism of the infusion pump and while the pumping mechanism applies a periodic compression force to the pumping segment.
Clause 2. The apparatus of Clause 1, wherein the pumping segment comprises two support keys fixedly attached to the pumping segment at opposite ends of the pumping segment.
Clause 3. The apparatus of Clause 2, wherein the apparatus comprises two rotating members spaced apart such that each keyway of each rotating member receives a respective one of the two support keys fixedly attached to the pumping segment when the pumping segment is loaded into the infusion pump.
Clause 4. The apparatus of Clause 3, wherein each of the two support keys comprises a swivel connector configured to allow rotation of the two support keys and the compressible tubing between the two support keys with respect to an upper tubing segment and lower tubing segment of the infusion set.
Clause 5. The apparatus of Clause 3, the two rotating members are connected together such that a rotation of a first rotating member of the two rotating members causes the two rotating members to rotate in unison.
Clause 6. The apparatus of Clause 5, wherein the two rotating members are injection molded such as to form a single part.
Clause 7. The apparatus of any one of Clauses 1-6, wherein the one or more rotating members are operatively coupled to a cam associated with the pumping mechanism such that the one or more rotating members rotate in coordination with a periodic compression action of the pumping mechanism upon the pumping segment.
Clause 8. The apparatus of any one of Clauses 1-7, wherein the one or more rotating members are configured to rock back and forth to cause the pumping segment to partially rotate back and forth about the central axis of the pumping segment.
Clause 9. The apparatus of Clause 8, further comprising: at least one gear operatively coupled to at least one of the one or more rotating members to cause the one or more rotating members to rotate in a first direction until a first threshold rotation is reached and then to rotate in an opposite direction until a second threshold rotation is reached in the opposite direction, and then to continually repeat the back and forth rotation until compression actions performed by the pumping mechanism upon the pumping segment are terminated.
Clause 10. The apparatus of Clause 9, wherein the at least one gear is configured to incrementally rotate the one or more rotating members in the first direction by a predetermined amount commensurate with a predetermined number of compression actions performed by the pumping mechanism until the first threshold rotation is reached and then to incrementally rotate the one or more rotating members in an opposite direction by the predetermined amount commensurate with another predetermined number of compression actions until the second threshold rotation is reached in the opposite direction, and then to continually repeat the back and forth rotation until the compression actions are terminated.
Clause 11. The apparatus of Clause 10, wherein the at least one gear comprises a rack and pinion system.
Clause 12. The apparatus of Clause 10, further comprising: a motor operatively coupled to the at least one gear to rotate the one or more rotating members independently of the pumping mechanism.
Clause 13. The apparatus of Clause 8, wherein the one or more rotating members comprise a gear or geared edge, the apparatus further comprising: a bi-directional rachet and pawl operatively coupled to at least one of the one or more rotating members to cause the one or more rotating members to incrementally rotate in a first direction by a predetermined amount commensurate with a predetermined number of compression actions performed by the pumping mechanism until a first threshold rotation is reached and then to incrementally rotate the one or more rotating members in an opposite direction by the predetermined amount commensurate with another predetermined number of compression actions until a second threshold rotation is reached in the opposite direction, and then to continually repeat the back and forth rotation until the compression actions are terminated.
Clause 14. The apparatus of any one of Clauses 1-13, wherein the one or more respective support keys comprise a support key having a shape configured to center a portion of the pumping segment constrained by the support key with respect to a sensor of the infusion pump, wherein the keyway corresponding to the support key comprises a matching shape to secure the support key, align the portion of the pumping segment with the sensor, align pumping segment with the pumping mechanism, and cause rotation of the support key to rotate the pumping segment.
Clause 15. An infusion set for loading into an infusion pump, comprising: a pumping segment comprising: a compressible tubing segment; and two support keys, fixed on opposing ends of the compressible tubing segment, wherein the two support keys comprises at least one gear, wherein the pumping segment is configured to be loaded into an infusion pump comprising one or more corresponding gears configure to, when the pumping segment is loaded into the infusion pump, interface with the at least one gear of the two support keys and configured to at least partially rotate the pumping segment about a central axis of the pumping segment by causing the at least one gear of the two support keys to at least partially rotate, wherein, when the pumping segment is loaded into the infusion pump, the pumping segment is constrained in a predetermined linear position between a rigid tubing support surface and a pumping mechanism of the infusion pump, while the pumping mechanism applies a periodic compression force to the compressible tubing segment of the pumping segment.
Clause 16. The infusion set of Clause 15, further comprising: an upper portion of tubing coupled to a first support key of the two support keys and a lower portion of tubing coupled to a second support key of the two support keys, wherein the lower portion of tubing is configured to fluidically couple to a catheter.
Clause 17. The infusion set of Clause 15 or Clause 16, wherein the compressible tubing segment is a portion of an infusion line passing through the two support keys, the two support keys being fixed to the infusion line by an adhesive.
Clause 18. The infusion set of any one of Clauses 15-17, wherein the at least one gear is formed by gear teeth being molded into at least a portion of at least one support key of the two support keys.
Clause 19. An method of operating a rotating infusion set in an infusion pump, comprising: receiving a pumping segment into the infusion pump, the pumping segment comprising a compressible tubing segment constrained by two support keys, fixed on opposing ends of the compressible tubing segment; receiving the two support keys into corresponding keyways of two rotating members of the infusion pump; and causing each rotating member to at least partially rotate the pumping segment about a central axis of the pumping segment by causing the one or more respective support keys to at least partially rotate, while constraining the pumping segment in a predetermined linear position between a rigid tubing support surface and a pumping mechanism of the infusion pump and while the pumping mechanism applies a periodic compression force to the pumping segment.
Clause 20. The method of Clause 19, further comprising: causing the pumping segment to partially rotate back and forth, periodically, in a reciprocal manner while an infusion is performed by the infusion pump.

Further Consideration:

In some embodiments, any of the clauses herein may depend from any one of the independent clauses or any one of the dependent clauses. In one aspect, any of the clauses (e.g., dependent or independent clauses) may be combined with any other one or more clauses (e.g., dependent or independent clauses). In one aspect, a claim may include some or all of the words (e.g., steps, operations, means or components) recited in a clause, a sentence, a phrase or a paragraph. In one aspect, a claim may include some or all of the words recited in one or more clauses, sentences, phrases or paragraphs. In one aspect, some of the words in each of the clauses, sentences, phrases or paragraphs may be removed. In one aspect, additional words or elements may be added to a clause, a sentence, a phrase or a paragraph. In one aspect, the subject technology may be implemented without utilizing some of the components, elements, functions or operations described herein. In one aspect, the subject technology may be implemented utilizing additional components, elements, functions or operations.

The previous description is provided to enable any person skilled in the art to practice the various aspects described herein. The previous description provides various examples of the subject technology, and the subject technology is not limited to these examples. Various modifications to these aspects will be readily apparent to those skilled in the art, and the generic principles defined herein may be applied to other aspects. Thus, the claims are not intended to be limited to the aspects shown herein, but is to be accorded the full scope consistent with the language claims, wherein reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention described herein.

The term website, as used herein, may include any aspect of a website, including one or more web pages, one or more servers used to host or store web related content, etc. Accordingly, the term website may be used interchangeably with the terms, web page and server. As used herein a “user interface” (also referred to as an interactive user interface, a graphical user interface or a UI) may refer to a network based interface including data fields and/or other control elements for receiving input signals or providing electronic information and/or for providing information to the user in response to any received input signals. Control elements may include dials, buttons, icons, selectable areas, or other perceivable indicia presented via the UI that, when interacted with (e.g., clicked, touched, selected, etc.), initiates an exchange of data for the device presenting the UI. A UI may be implemented in whole or in part using technologies such as hyper-text mark-up language (HTML), FLASH™, JAVA™, .NET™, web services, or rich site summary (RSS). In some implementations, a UI may be included in a stand-alone client (for example, thick client, fat client) configured to communicate (e.g., send or receive data) in accordance with one or more of the aspects described. The communication may be to or from a medical device, diagnostic device, monitoring device, or server in communication therewith.

The predicate words “configured to”, “operable to”, and “programmed to” do not imply any particular tangible or intangible modification of a subject, but, rather, are intended to be used interchangeably. For example, a processor configured to monitor and control an operation or a component, may also mean the processor being programmed to monitor and control the operation or the processor being operable to monitor and control the operation. Likewise, a processor configured to execute code can be construed as a processor programmed to execute code or operable to execute code.

The term automatic, as used herein, may include performance by a computer or machine without user intervention; for example, by instructions responsive to a predicate action by the computer or machine or other initiation mechanism. The word “example” is used herein to mean “serving as an example or illustration.” Any aspect or design described herein as “example” is not necessarily to be construed as preferred or advantageous over other aspects or designs.

As used herein, the terms “correspond” or “corresponding” encompasses a structural, functional, quantitative and/or qualitative correlation or relationship between two or more objects, data sets, information and/or the like, preferably where the correspondence or relationship may be used to translate one or more of the two or more objects, data sets, information and/or the like so to appear to be the same or equal. Correspondence may be assessed using one or more of a threshold, a value range, fuzzy logic, pattern matching, a machine learning assessment model, or combinations thereof.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “implementation” does not imply that such implementation is essential to the subject technology or that such implementation applies to all configurations of the subject technology. A disclosure relating to an implementation may apply to all implementations, or one or more implementations. An implementation may provide one or more examples. A phrase such as an “implementation” may refer to one or more implementations and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such as a “configuration” may refer to one or more configurations and vice versa.

1. An apparatus for preserving an elasticity of a pumping segment of an infusion set into an infusion pump, comprising:
a rigid tubing support surface; and
one or more rotating members each comprising a keyway configured to receive one or more respective support keys fixedly attached to the pumping segment such that a rotation of a compressible tubing of the pumping segment is prevented from rotating with respect to the one or more respective support keys,
the one or more rotating members being configured to, when each support key of the pumping segment is received into the corresponding keyway, at least partially rotate the pumping segment and the compressible tubing about a central axis of the pumping segment by causing the one or more respective support keys to at least partially rotate, while constraining the pumping segment in a predetermined linear position between the rigid tubing support surface and a pumping mechanism of the infusion pump and while the pumping mechanism applies a periodic compression force to the pumping segment.

2. The apparatus of claim 1, wherein the pumping segment comprises two support keys fixedly attached to the pumping segment at opposite ends of the pumping segment.

3. The apparatus of claim 2, wherein the apparatus comprises two rotating members spaced apart such that each keyway of each rotating member receives a respective one of the two support keys fixedly attached to the pumping segment when the pumping segment is loaded into the infusion pump.

4. The apparatus of claim 3, wherein each of the two support keys comprises a swivel connector configured to allow rotation of the two support keys and the compressible tubing between the two support keys with respect to an upper tubing segment and lower tubing segment of the infusion set.

5. The apparatus of claim 3, the two rotating members are connected together such that a rotation of a first rotating member of the two rotating members causes the two rotating members to rotate in unison.

6. The apparatus of claim 5, wherein the two rotating members are injection molded such as to form a single part.

7. The apparatus of claim 1, wherein the one or more rotating members are operatively coupled to a cam associated with the pumping mechanism such that the one or more rotating members rotate in coordination with a periodic compression action of the pumping mechanism upon the pumping segment.

8. The apparatus of claim 1, wherein the one or more rotating members are configured to rock back and forth to cause the pumping segment to partially rotate back and forth about the central axis of the pumping segment.

9. The apparatus of claim 8, further comprising:
at least one gear operatively coupled to at least one of the one or more rotating members to cause the one or more rotating members to rotate in a first direction until a first threshold rotation is reached and then to rotate in an opposite direction until a second threshold rotation is reached in the opposite direction, and then to continually repeat the back and forth rotation until compression actions performed by the pumping mechanism upon the pumping segment are terminated.

10. The apparatus of claim 9, wherein the at least one gear is configured to incrementally rotate the one or more rotating members in the first direction by a predetermined amount commensurate with a predetermined number of compression actions performed by the pumping mechanism until the first threshold rotation is reached and then to incrementally rotate the one or more rotating members in an opposite direction by the predetermined amount commensurate with another predetermined number of compression actions until the second threshold rotation is reached in the opposite direction, and then to continually repeat the back and forth rotation until the compression actions are terminated.

11. The apparatus of claim 10, wherein the at least one gear comprises a rack and pinion system.

12. The apparatus of claim 10, further comprising:
a motor operatively coupled to the at least one gear to rotate the one or more rotating members independently of the pumping mechanism.

13. The apparatus of claim 8, wherein the one or more rotating members comprise a gear or geared edge, the apparatus further comprising:
a bi-directional rachet and pawl operatively coupled to at least one of the one or more rotating members to cause the one or more rotating members to incrementally rotate in a first direction by a predetermined amount commensurate with a predetermined number of compression actions performed by the pumping mechanism until a first threshold rotation is reached and then to incrementally rotate the one or more rotating members in an opposite direction by the predetermined amount commensurate with another predetermined number of compression actions until a second threshold rotation is reached in the opposite direction, and then to continually repeat the back and forth rotation until the compression actions are terminated.

14. The apparatus of claim 1, wherein the one or more respective support keys comprise a support key having a shape configured to center a portion of the pumping segment constrained by the support key with respect to a sensor of the infusion pump, wherein the keyway corresponding to the support key comprises a matching shape to secure the support key, align the portion of the pumping segment with the sensor, align pumping segment with the pumping mechanism, and cause rotation of the support key to rotate the pumping segment.

15. An infusion set for loading into an infusion pump, comprising:
a pumping segment comprising:
a compressible tubing segment; and
two support keys, fixed on opposing ends of the compressible tubing segment,
wherein the two support keys comprises at least one gear,
wherein the pumping segment is configured to be loaded into an infusion pump comprising one or more corresponding gears configure to, when the pumping segment is loaded into the infusion pump, interface with the at least one gear of the two support keys and configured to at least partially rotate the pumping segment about a central axis of the pumping segment by causing the at least one gear of the two support keys to at least partially rotate,
wherein, when the pumping segment is loaded into the infusion pump, the pumping segment is constrained in a predetermined linear position between a rigid tubing support surface and a pumping mechanism of the infusion pump, while the pumping mechanism applies a periodic compression force to the compressible tubing segment of the pumping segment.

16. The infusion set of claim 15, further comprising:
an upper portion of tubing coupled to a first support key of the two support keys and a lower portion of tubing coupled to a second support key of the two support keys, wherein the lower portion of tubing is configured to fluidically couple to a catheter.

17. The infusion set of claim 15, wherein the compressible tubing segment is a portion of an infusion line passing through the two support keys, the two support keys being fixed to the infusion line by an adhesive.

18. The infusion set of claim 15, wherein the at least one gear is formed by gear teeth being molded into at least a portion of at least one support key of the two support keys.

19. An method of operating a rotating infusion set in an infusion pump, comprising:
receiving a pumping segment into the infusion pump, the pumping segment comprising a compressible tubing segment constrained by two support keys, fixed on opposing ends of the compressible tubing segment;
receiving the two support keys into corresponding keyways of two rotating members of the infusion pump; and
causing each rotating member to at least partially rotate the pumping segment about a central axis of the pumping segment by causing the one or more respective support keys to at least partially rotate, while constraining the pumping segment in a predetermined linear position between a rigid tubing support surface and a pumping mechanism of the infusion pump and while the pumping mechanism applies a periodic compression force to the pumping segment.

20. The method of claim 19, further comprising:
causing the pumping segment to partially rotate back and forth, periodically, in a reciprocal manner while an infusion is performed by the infusion pump.
